(19) United States
(12) Patent Application Publication
(10) Pub. No.: US 2005/0033031 A1
(43) Pub. Date: Feb. 10, 2005

(54) Methods for humanizing rabbit monoclonal antibodies

(76) Inventor: Fernando Jose Rebelo Do Couto, Pleasanton, CA (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/637,317

(22) Filed: Aug. 7, 2003

(51) Int. Cl. C12Q 1/68; C07H 21/04; C07K 16/44; C12N 5/06

(52) U.S. Cl. 530/388.15; 435/69.1; 435/328; 435/320.1; 536/23.53

(57) ABSTRACT

The invention provides a method for humanizing a rabbit monoclonal antibody. In general, the method involves comparing an amino acid sequences of a parent rabbit antibody to the amino acid sequences of a similar human antibody, and altering the amino acid sequence of the parent rabbit antibody such its framework regions are more similar in sequence to the equivalent framework regions of the similar human antibody. In many embodiments, amino acids in the parent rabbit antibody that are not CDR contact residues, interchain contact residues, or buried residues, are not modified. The invention further provides nucleic acids encoding the subject antibodies, as well as vectors and host cells comprising the nucleic acids and methods for producing a subject antibody. The subject antibodies, nucleic acid compositions and kits find use in a variety of applications, including diagnostics and therapeutic treatment and research of conditions and diseases.

### FIELD OF THE INVENTION

The field of this invention is antibodies, particularly methods for humanizing rabbit monoclonal antibodies.

### BACKGROUND OF THE INVENTION

Monoclonal antibodies, their conjugates and derivatives have the potential to become one of the main therapeutic agents of the future, because of their ability to target virtually any molecule with exquisite specificity. Though this potential was recognized very early, the first attempts to fulfill it were disappointing mainly because monoclonal antibodies used in therapy elicited a strong immune response in patients (Schroff, 1985 Cancer Res 45:879-85, Shawler. J Immunol 1985 135:1530-5) usually following a single low dose injection (Dillman, Cancer Biother 1994 9:17-28). Scientists predicted that human antibodies would not cause such adverse immune responses but there was no hybridoma technology that could produce human monoclonal antibodies. Alternative technologies to make human antibodies using, for example, phage display and transgenic animals were later developed. Nevertheless, there was still a need to devise means to overcome the immunogenicity of rodent antibodies because they already had useful and well characterized antigen specifities. In addition, certain useful antigen binding characteristics might be rare and difficult or impossible to reproduce outside the rodent immune system.

The immunogenicity of antibodies depends on many factors, including the method of administration, the number of injections, the dosage, the nature of the conjugation, the specific fragment utilized, the state of aggregation and the nature of the antigen (e.g., Kuus-Reichel, Clin Diagn Lab Immunol 1994 1:365-72). Many or most of these factors can be manipulated in order to decrease an immune response but if the original antibody sequence is recognized as “dangerous” or “foreign” chances are that sooner or later a strong immune response will prevent the use of that antibody in therapy.

The engineering of chimeric antibodies, which combine rodent FV fragments with human FC fragments (e.g., Boulianne Nature 1984 312:643-6) reduced immunogenicity problems considerably (e.g., LoBuglio, Proc Natl Acad Sci 1989 86:4220-4) while at the same time making it possible to make use of human effector domains in therapy (Clark, Immunol Today 2000 21:397-402). Even further, humanized antibodies were engineered in which the rodent sequence of the FV itself is engineered to be as close to a human sequence as possible while preserving at least the original CDRs (e.g., Riechmann, Nature 1988 332:323-7). Humanized rodent antibodies also showed much reduced immunogenicity in human patients (Moreland, Arthritis Rheum 1993 36:307-18) though some humanized antibodies are still immunogenic to a large proportion of patients, most likely because the rodent CDRs themselves are immunogenic (Ritter, Cancer Res 2001 61:6851-9; Welt, Clin Cancer Res 2003 9:1338-46).

Today (2003) a dozen or so monoclonal antibody products are used in the clinic, generating in the order of $2 billion in revenues, while many more dozens are in advanced clinical trials. Many of these clinical antibodies are chimeric, humanized or human and most were originally mouse monoclonal antibodies.

The preponderance of mouse antibodies is not due to their superiority over those of other species, but rather due the absence, until now, of a non-rodent hybridoma technology. This situation has now changed. Rabbit is one of the best sources of high quality antibodies, due to its robust immune response and its propensity to produce very high-affinity antibodies to a wide range of epitopes. Recently it has become possible to generate rabbit monoclonal antibodies using conventional fusion methods (Spieker-Polet, Proc Natl Acad Sci 1995 92:9348-52). Therefore, very high quality monoclonal rabbit antibodies can be generated.

If used in therapy, however, rabbit antibodies, like mouse antibodies, are expected to elicit strong immune responses that will prevent prolonged repeated administrations. Thus, the same need to make chimeric and humanized rabbit antibodies must be fulfilled before they can be used clinically. However, the methods that are used to make chimeric and humanized rodent antibodies cannot be used for many rabbit antibodies for the following reasons:

Firstly, many rabbit kappa chains have a disulfide bond between the variable region and the constant region. This structural feature causes a problem for making chimeric and humanized antibodies that has not, to our knowledge been addressed. Chains of the Kappa-1 (K-1) isotype are the most commonly used rabbit antibody light chains. Three of the five commonly found K-1 allotypes (b4, b5, and b6) have a cysteine in framework 3 at position 80 in the variable region (VK). The side chain of this residue is exposed and makes a disulfide bond to another cysteine residue in the constant kappa domain. Rabbits of the fourth commonly found K-1 allotype, b9, also have the extra disulfide, but in this case the cysteine residue in the variable region occupies the last position, residue 108, of VK in framework 4. The kappa chains of human and rodent antibodies do not have this extra disulfide bond in the kappa chains. Therefore, if one were to construct a chimeric or humanized antibody following one of the many known methods, by joining the rabbit variable kappa domain to the human constant kappa domain a cysteine residue would remain unpaired in the variable kappa region. This would most likely cause protein folding and expression problems and even if a high yield of correctly folded antibody were obtained, the unpaired cysteine residue would most likely cause a fraction of the antibodies to dimerize via their VK cys residues, which is usually undesirable.

Secondly, many rabbit heavy chain variable regions are short by one or two amino-acid residues in the loop between beta strands D and E, relative to human and murine residues. Moreover, many heavy and light chains are also short by one residue at the N-terminus relative to human and murine chains. While these two regions are not usually in contact with antigen in human and murine antibodies they are, nevertheless, very near the CDRs and often make contact with the CDR residues. Obviously, for these rabbit antibody chains one cannot find a corresponding homologous human residue at the described positions because the positions do not exist.

Thirdly, many rabbit VH chains have extra paired cysteines relative to the murine and human counterparts. For example in some rabbit chains in addition to cys22-cys92 “normal” disulfide bond there is also a cys21-cys79 S—S bond as well as another bond between the last cys residue of CDR H1 and the first residue of CDR H2. Also frequently one also finds pairs of cysteine residues in the VK L3 CDR. By modeling the rabbit antibody structures by homology to know structures one can see that the cysteine pairs are spacially placed in positions that allow disulfide bond formation.

Finally, many rabbit antibody CDRs do not belong to any previously known canonical structures. In particular the VK CDR L3 is often much longer than previously known L3 CDRs from human or mouse antibodies. The lack of previous structural knowledge of rabbit CDRs makes it unlikely that they can be modeled accurately.

As such, because of the peculiarities of rabbit monoclonal antibodies, current methods for humanizing rodent antibodies cannot be readily used to humanize rabbit monoclonal antibodies. Accordingly, there is an urgent need for methods for humanizing rabbit antibodies. The present invention addresses this, and other, needs.

Literature

References of interest include: U.S. Pat. Nos. 6,331,415 B1, 5,225,539, 6,342,587, 4,816,567, 5,639,641, 6,180,370, 5,693,762, 4,816,397, 5,693,761, 5,530,101, 5,585,089, 6,329,551, and publications Morea et al., Methods 20: 267-279 (2000), Ann. Allergy Asthma Immunol. 81:105-119 (1998), Rader et al,. J. Biol. Chem. 276:13668-13676 (2000), Steinberger et al., J. Bio. Chem. 275: 36073-36078 (2000), Roguska et al., Proc. Natl. Acad. Sci. 91: 969-973 (1994), Delagrave et al., Prot. Eng. 12: 357-362 (1999), Rogusca et al., Prot. Eng. 9: 895-904 (1996), Knight and Becker, Cell 60: 963-970 (1990); Becker and Knight, Cell 63:987-997 (1990) and Popkov, J Mol Biol 325:325-35 (2003).

### SUMMARY OF THE INVENTION

The invention provides a method for humanizing a rabbit monoclonal antibody. In general, the method involves comparing the amino acid sequences of a parent rabbit antibody to the amino acid sequences of a similar human antibody, and altering the amino acid sequence of the parent rabbit antibody such its framework (FW) regions are more similar in sequence to the equivalent framework regions of the similar human antibody. In certain embodiments, the FW1 regions from both the heavy and light chains of a rabbit antibody may be replaced with corresponding FW1 regions of a similar human antibody, which, in most embodiments adds at least one amino acid (i.e., 1, 2 or 3 or more amino acids) to the humanized antibody sequence, as compared to the parent antibody sequence. In other embodiments, the entire D-E loop of the heavy chain variable domain of the rabbit antibody may be replaced by the corresponding loop of the similar human antibody, which, in many embodiments, adds at least one amino acid (i.e., 1, 2 or 3 or more amino acids). In certain other embodiments, if a cys80 is present in the light chain of the antibody, that amino acid is replaced by the corresponding amino acid, or by replacing the corresponding E-F loop of the human antibody. Finally, cysteine pairs that are deemed to be close to each other may also be changed. In many embodiments, amino acids in the parent rabbit antibody that are CDR contact residues, interchain contact residues, or buried residues, are not modified. The invention further provides nucleic acids encoding the subject antibodies, as well as vectors and host cells comprising the nucleic acids and methods for producing a subject antibody. The subject antibodies, nucleic acid compositions and kits find use in a variety of applications, including diagnostics and therapeutic treatment and research of conditions and diseases.

In many embodiments, the amino acids that are involved in complementarity determining region (CDR) contacts are selected from the amino acids at positions 1, 2, 4, 24, 27, 28, 29, 30, 36, 38, 40, 46, 48, 49, 66, 67, 68, 69, 71, 73, 78, 80, 82, 86, 92, 93 and 94 in the heavy chain variable domain and the amino acids at positions 1, 2, 3, 4, 5, 7, 22, 23, 35, 45, 48, 49, 58, 60, 62, 66, 67, 69, 70, 71 and 88 in a kappa light chain variable domain.

In many embodiments, the amino acids that are involved in interchain contacts are selected from the amino acids at positions 37, 39, 43, 44, 45, 47, 91, 103 and 105 in the heavy chain variable domain and the amino acids at positions 36, 38, 43, 44, 46, 85, 87, 98 and 100 in the kappa light chain variable domain.

In many embodiments the buried residues are selected from the amino acids at positions 6, 9, 12, 18, 20, 22, 76, 82c, 88, 90, 107, 109 and 111 in the heavy chain variable domain and the amino acids at positions 6, 11, 13, 19, 21, 37, 47, 61, 73, 75, 78, 82, 83, 84, 86, 102, 104 and 106 in the kappa light chain variable domain.

These and other advantages and features of the invention will become apparent to those persons skilled in the art upon reading the details of the invention as more fully described below.

### BRIEF DESCRIPTION OF THE DRAWINGS

FIG. 1 is a figure that schematically illustrates some embodiments of the invention.

FIG. 2 is a multiple sequence alignment of variable kappa (top) and variable heavy (bottom) chains cloned from different rabbit hybridomas. Standard numbering, and the position of the beta strands (A, A′, B, C, C′, D, E, F, G) is provided on the top of each alignment. These positions are based on the literature (Chothia J Mol Biol 1998 278:457-79). UP4_{—}31: SEQ ID NO:1; UP4_{—}29: SEQ ID NO:2; UP4_{—}23: SEQ ID NO:3; CALK_VK: SEQ ID NO:4; CD79_A: SEQ ID NO:5; UP3_{—}4_V: SEQ ID NO:6; CS1_{—}108: SEQ ID NO:7; CS1_{—}115: SEQ ID NO:8; PLAP_VK: SEQ ID NO:9; B1_VK: SEQ ID NO:10; DEW76: SEQ ID NO:11; DEW148: SEQ ID NO:12; B1_VH: SEQ ID NO:13; DEW73: SEQ ID NO:14; DEW70: SEQ ID NO:15; and KabX: SEQ ID NO:16

FIG. 3 is a multiple sequence alignment showing humanization of the anti-integrin beta-6 rabbit monoclonal antibody B1. The original B1 VK and VH sequences are shown aligned with their respective closest human target germline sequences and final humanized chain sequences. The alignments are broken at the end of CDRs and frameworks (FRs) for ease of reading. The standard numbering is shown on the top line. Shaded regions in the numbering line indicated where the CDRs are. The other shaded areas represent framework positions that differ from the original rabbit sequences. Black cells are deletions in the rabbit sequence relative to the human counterparts. Part of the human VK CDR3 and the complete VH CDR3 are not shown because they are not precisely encoded in the human germline. B1VK: SEQ ID NO:17; Hu_L12_JK4: SEQ ID NO17; B1_VK_HZ1: SEQ ID NO:20; B1VH: SEQ ID NO:21; B1VH_HZ1: SEQ ID NO:22.

FIG. 4 is a schematic representation of a Fab antibody fragment showing the “extra” disulfide bond between VK and CK that is present in some rabbit but not in human or murine kappa chains.

FIG. 5 is a schematic representation of the structure of a rabbit VH domain showing the positions of the three CDRs and the D-E loop.

### DEFINITIONS

Before the present subject invention is described further, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an antibody” includes a plurality of such antibodies and reference to “a framework region” includes reference to one or more framework regions and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “host organism” means any animal that produces antibodies that have a variable regions that is structurally similar to those of rabbits. Exemplary host organisms include humans, a mice, rats, etc.

An amino acid residue that is in “close contact”, “close proximity” or “in close proximity to” another amino acid residue is an amino acid residue that is has a side chain that is close to, i.e., within 7, 6, 5 or 4 Angstroms of, a side chain of another amino acid. For example, an amino acid that are proximal to a CDR is a non-CDR amino acid that has a side chain that is close to a side chain of an amino acid in a CDR.

A “variable region” of a heavy or light antibody chain is an N-terminal mature domain of the chains. All domains, CDRs and residue numbers are assigned on the basis of sequence alignments and structural knowledge. Identification and numbering of framework residues is as described in by Chothia and others (Chothia Structural determinants in the sequences of immunoglobulin variable domain. J Mol Biol 1998; 278:457-79).

VH is the variable domain of an antibody heavy chain. VL is the variable domain of an antibody light chain, which could be of the kappa (K) or of the lambda isotype. K-1 antibodies have the kappa-1 isotype whereas K-2 antibodies have the kappa-2 isotype and VL is the variable lambda light chain.

A “buried residue” is an amino acid residue whose side chain has less than 50% relative solvent accessibility, which is calculated as the percentage of the solvent accessibility relative to that of the same residue, X, placed in an extended GGXGG (SEQ ID NO:23) peptide. Methods for calculating solvent accessibility are well known in the art (Connolly 1983 J. appl. Crystallogr, 16, 548-558).

The terms “antibody” and “immunoglobulin” are used interchangeably herein. These terms are well understood by those in the field, and refer to a protein consisting of one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the terms are Fab′, Fv, F(ab′)₂, and or other antibody fragments that retain specific binding to antigen.

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986),).

An immunoglobulin light or heavy chain variable region consists of a “framework” region (FR) interrupted by three hypervariable regions, also called “complementarity determining regions” or CDRs. The extent of the framework region and CDRs have been precisely defined (see, “Sequences of Proteins of Immunological Interest,” E. Kabat et al., U.S. Department of Health and Human Services, (1991)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

Chimeric antibodies are antibodies whose light and heavy chain genes have been constructed, typically by genetic engineering, from antibody variable and constant region genes belonging to different species. For example, the variable segments of the genes from a rabbit monoclonal antibody may be joined to human constant segments, such as gamma 1 and gamma 3. An example of a therapeutic chimeric antibody is a hybrid protein composed of the variable or antigen-binding domain from a rabbit antibody and the constant or effector domain from a human antibody (e.g., the anti-Tac chimeric antibody made by the cells of A.T.C.C. deposit Accession No. CRL 9688), although other mammalian species may be used.

As used herein, the term “humanized antibody” or “humanized immunoglobulin” refers to an antibody comprising one or more CDRs from a rabbit antibody; and a rabbit framework region that contains amino acid substitutions and/or deletions and/or insertions that are based on a human antibody sequence. The rabbit immunoglobulin providing the CDRs is called the “parent” or “acceptor” and the human antibody providing the framework changes is called the “donor”. Constant regions need not be present, but if they are, they are usually substantially identical to human antibody constant regions, i.e., at least about 85-90%, preferably about 95% or more identical. Hence, in some embodiments, a full length humanized rabbit heavy or light chain immunoglobulin contains a human constant region, rabbit CDRs, and a substantially rabbit framework that has a number of “humanizing” amino acid alterations, which will be described in detail below. In many embodiments, a “humanized antibody” is an antibody comprising a humanized variable light chain and/or a humanized variable heavy chain. For example, a humanized antibody would not encompass a typical chimeric antibody as defined above, e.g., because the entire variable region of a chimeric antibody is non-human. A modified antibody that has been “humanized” by the process of “humanization” binds to the same antigen as the parent antibody that provides the CDRs and is usually less immunogenic in humans, as compared to the parent antibody.

It is understood that the humanized antibodies designed and produced by the present method may have additional conservative amino acid substitutions which have substantially no effect on antigen binding or other antibody functions. By conservative substitutions is intended combinations such as those from the following groups: gly, ala; val, ile, leu; asp, glu; asn, gin; ser, thr; lys, arg; and phe, tyr. Amino acids that are not present in the same group are “substantially different” amino acids.

As used herein, the terms “determining,” “measuring,” and “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

As used herein the term “isolated,” when used in the context of an isolated antibody, refers to an antibody of interest that is at least 60% free, at least 75% free, at least 90% free, at least 95% free, at least 98% free, and even at least 99% free from other components with which the antibody is associated with prior to purification.

The terms “treatment” “treating” and the like are used herein to refer to any treatment of any disease or condition in a mammal, e.g. particularly a human or a mouse, and includes: a) preventing a disease, condition, or symptom of a disease or condition from occurring in a subject which may be predisposed to the disease but has not yet been diagnosed as having it; b) inhibiting a disease, condition, or symptom of a disease or condition, e.g., arresting its development and/or delaying its onset or manifestation in the patient; and/or c) relieving a disease, condition, or symptom of a disease or condition, e.g., causing regression of the condition or disease and/or its symptoms.

The terms “subject,” “host,” “patient,” and “individual” are used interchangeably herein to refer to any mammalian subject for whom diagnosis or therapy is desired, particularly humans. Other subjects may include cattle, dogs, cats, guinea pigs, rabbits, rats, mice, horses, and so on.

### DETAILED DESCRIPTION OF PREFERRED EMBODIMENTS

The invention provides a method for humanizing a rabbit monoclonal antibody. In general, the method involves comparing the amino acid sequences of a parent rabbit antibody to the amino acid sequences of a similar human antibody, and altering the amino acid sequence of the parent rabbit antibody such its framework regions are more similar in sequence to the equivalent framework regions of the similar human antibody. In many embodiments, amino acids in the parent rabbit antibody that are not CDR contact residues, interchain contact residues, or buried residues, are not modified. The invention further provides nucleic acids encoding the subject antibodies, as well as vectors and host cells comprising the nucleic acids and methods for producing a subject antibody. The subject antibodies, nucleic acid compositions and kits find use in a variety of applications, including diagnostics and therapeutic treatment and research of conditions and diseases.

In further describing the subject invention, methods of humanizing a rabbit monoclonal antibody are discussed first, followed by a description of nucleic acids encoding an antibody humanized by the subject methods and a review of the methods and representative applications in which the subject systems find use.

METHODS FOR HUMANIZING RABBIT MONOCLONAL ANTIBODIES

The subject invention provides methods for humanizing a rabbit antibody. The methods generally involve altering certain amino acids of the heavy and light chain variable domain framework regions of the antibody such that the framework regions of the humanized rabbit antibody are more similar in sequence to those of a human antibody. These humanized rabbit antibodies are usually less immunogenic in a human host than an unmodified, parent rabbit antibody, while retaining specific binding to an antigen, usually a predetermined antigen, with high affinity. In other words, the subject methods may be used to produce a humanized rabbit antibody, where a humanized rabbit antibody is usually less immunogenic than a parent rabbit antibody in a human host, and has a binding affinity of at least about 10⁷ M⁻¹, preferably 10⁸ M⁻¹ to 10¹⁰ M⁻¹, or higher to an antigen to which the unmodified parent antibody binds. In many embodiments, the alterations are made only to amino acids that are not thought to be structurally important, which structurally important amino acids may be CDR contact residues, interchain contact residues, or buried residues, as described herein in great detail. In certain embodiments, the FW1 regions from both the heavy and light chains of a rabbit antibody may be replaced with corresponding FW1 regions of a similar human antibody, which, in most embodiments adds at least one amino acid (i.e., 1, 2 or 3 or more amino acids) to the humanized antibody sequence, as compared to the parent antibody sequence. In other embodiments, the entire D-E loop of the heavy chain variable domain of the rabbit antibody may be replaced by the corresponding loop of a similar human antibody, which, in many embodiments, adds at least one amino acid (i.e., 1, 2 or 3 or more amino acids). In certain other embodiments, if a cys80 is present in the light chain of the antibody, that amino acid is replaced by the corresponding amino acid, or by replacing the E-F loop with that of a similar of the human antibody. Finally, cysteine pairs that are deemed to be close to each other may also be changed.

The subject methods find use in humanizing all rabbit antibodies. However, in particular embodiments, the subject methods find particular use in humanizing rabbit antibodies that have a light chain CDR3 that is a “long” CDR3, which is usually 10, 11, 12, 13, 14 or 15 residues in length as compared to human and mouse light chain CDR3s, which CDR is usually 6 residues long.

The humanized rabbit antibodies can be produced economically in large quantities and find use, for example, in the diagnosis and treatment of various human and mouse disorders by a variety of techniques.

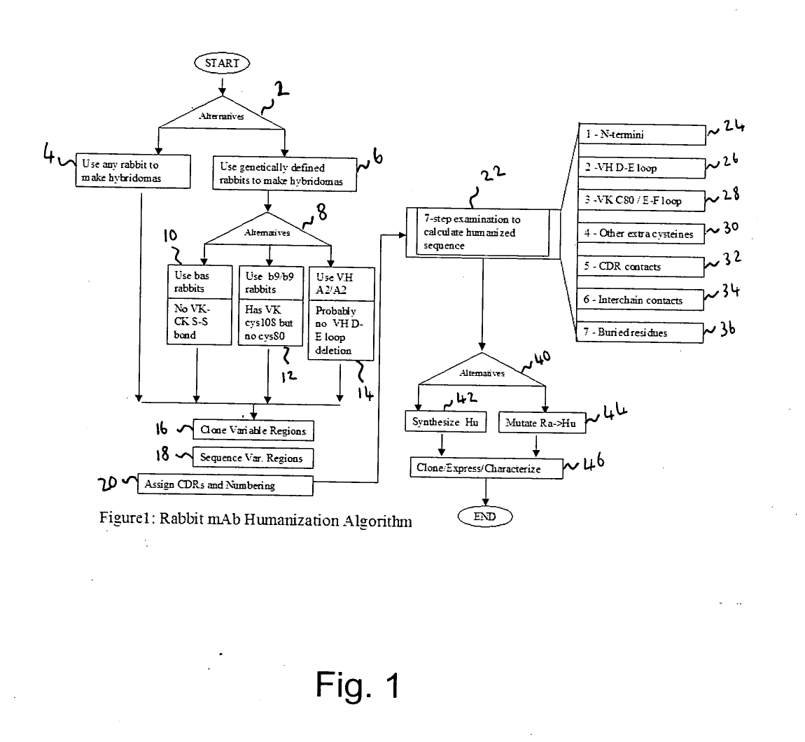

FIG. 1 is a flow chart showing a general overview of certain embodiments of the subject methods. With specific reference to FIG. 1, these methods start by making a choice of rabbits 2 for immunization and monoclonal antibody production. Either any rabbit may be used 4, or, in certain embodiments, a genetically defined rabbit may be used 6. Certain types of genetically defined rabbits may be chosen 8, primarily on the basis of whether antibodies with various S-S bonds, or a VH D-E loop are desired. For example, bas rabbits 10 may be used to produce an antibody with no VK-CK disulphide bond, b9/b9 rabbits 12 may be used to produce an antibody with a cys108 but no cys80, or A2/A2 rabbits 14 may be used to produce an antibody that usually has no VH D-E loop deletion. Once a suitable monoclonal antibody has been identified and produced, the nucleic acids encoding variable regions of the antibody are, in many embodiments, cloned 16, sequenced 20, and the amino acid sequence of the variable regions of the antibody are determined. The CDRs are identified and the amino acids are numbered 20, usually according to the scheme of Chothia, supra or Kabat supra. Then, a similar human antibody is usually identified and the sequence of the rabbit antibody is changed according to the following steps: a) replacing the N-termini of the heavy and/or light chain variable domains of the rabbit antibody with those of the similar human antibody 24; b) replacing the entire VH D-E loop of the rabbit antibody with that of the similar human antibody 26; c) replacing a cys80 of the light chain of the rabbit antibody with a corresponding amino acid from the similar human antibody, or, in other embodiments, replacing the entire E-F loop of the rabbit antibody with that the similar human antibody 28; d) removing cysteine pairs from the antibody, where the cysteine pairs are thought to be in close proximity in the antibody 30, and e) not changing any residues that are deemed to be involved in CDR contacts 32, interchain contacts 34, or are buried residues 36. After the sequence of the variable domains of the humanized rabbit antibody has been designed 22, nucleic acids encoding the variable domains may be made by two alternative exemplary approaches 40, which approaches are to synthesize the variable domain nucleic acids de novo 42, or alter the parent rabbit monoclonal antibody variable domain nucleic acids such that they encode the humanized variable domains 44. Subsequent to their making, the variable domain nucleic acids may be cloned into an appropriate vector to provide for antibody production, expressed in a cell, and the encoding antibodies characterized 46.

Rabbit Immunoglobulin VH and VL Chain Sequences

As a first step in the subject method, the amino acid sequence of a rabbit monoclonal antibody (a “parental” antibody) is obtained. In many embodiments, the specificity for the monoclonal antibody is known, however, in certain embodiments, its specificity is unknown.

Rabbit antibodies are generated by immunizing a rabbit with an antigen or mixture of antigens, and rabbit immunoglobulin heavy and light chain variable domain sequences are usually determined by sequencing the nucleic acids (particularly cDNAs) that encode them. As discussed above, depending on the desired sequence characteristics of a parent rabbit antibody, a variety of genotypes of rabbit may be used in the subject method. In general, any rabbit, including rabbits having the basilea (bas) and b9/b9 genotypes and A2/A2 may be used. These nucleic acids may be isolated from any antibody-producing cell or mixture of cells e.g. bone marrow, spleen, etc., from an immunized rabbit or a hybridoma cell producing a rabbit antibody. In most embodiments, antibody-encoding nucleic acids are isolated from these cells using standard molecular biology techniques such as polymerase chain reaction (PCR) or reverse transcription PCR (RT-PCR) (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.).

In many embodiments nucleic acids encoding the VH and VL domains of a parent rabbit antibody are isolated from a rabbit antibody-producing hybridoma cell. In order to produce rabbit antibody-producing hybridoma lines, rabbits are immunized with an antigen and once a specific immune response of the rabbit has been established, cells from the spleen of the immunized rabbit are fused with a plasmacytoma cell line such as 240E (Spieker-Polet et al, Proc. Natl. Acad. Sci. 92: 9348-9352, 1995). After fusion, the cells are grown in medium containing hypoxanthine, aminopterin, and thymidine (HAT) to select for hybridoma growth, and after 2-3 weeks, hybridoma colonies appear. Supernatants from these cultured hybridoma cells are screened for antibody secretion by enzyme-linked immunosorbent assay (ELISA) and positive clones secreting monoclonal antibodies specific for the antigen can be selected and expanded according to standard procedures (Harlow et al,. Antibodies: A Laboratory Manual, First Edition (1988) Cold spring Harbor, N.Y.; and Spieker-Polet et al., supra).

In other embodiments, the parent rabbit antibody-encoding nucleic acids are isolated from cells by any known method. Exemplary methods include 1) performing flow cytometry of cell populations obtained from rabbit spleen, bone marrow, lymph node or other lymph organs followed by single-cell plating, e.g., through incubating the cells with labeled anti-rabbit IgG and sorting the labeled cells using a FACSVantage SE cell sorter (Becton-Dickinson, San Jose, Calif.); and 2) plating of plasma cells in multi-well plates at limiting dilutions. Cells can be directly sorted into 96-well or 384-well plates containing RT-PCR buffer, and subjected to RT-PCR with nested primers specific for the IgG heavy and light chains. As an alternative to cell sorting, limiting dilution cell plating can be used in order to obtain single B cells.

The methods of the invention, although appropriate for modifying any rabbit antibody, are usually used to modify a “natural” antibody, where the heavy and light immunoglobulins of the antibody have been naturally selected by the immune system of a rabbit, as opposed to “unnaturally” paired antibodies made by e.g. phage display. The antibodies described herein are generally not associated, i.e., operably linked, to viral sequences, e.g., viral coat protein sequences.

Sequence Comparison

Once the amino acid sequences of the VH and VL domains of the parent rabbit antibody have been determined, the amino acids are usually numbered using a suitable numbering system, such as that provided by Chothia 1998, supra or Kabat supra, and the CDR and/or framework residues are usually identified. The sequences are usually compared to a database of sequences of human immunoglobulin sequences, usually germline sequences, in order to identify a similar human antibody. This similar human antibody may be termed a “donor” antibody because amino acids will generally be transferred from the human antibody to the parent rabbit antibody. Typically, the parent rabbit VL or VH sequences will be compared to the database using a suitable comparison program, e.g., BLASTP, FASTP, at default settings, and a similar human antibody is identified as having one of the 10 (or, in some embodiments, one of the 3 or the top 1) most similar variable domains (VL or VH) in terms of amino acid sequence identity (either by percent identity or P-value) to a parent variable domain sequence. The selected donor antibody variable regions will typically have at least about 55%, at least about 65%, at least about 75%, at least about 80%, at least about 85%, at least about 90%, or at least about 95% amino acid sequence identity in the framework region to the parent framework region. In some embodiments, sequences are compared to an amino acid sequence that is not stored in a database, e.g. to the sequence of a newly sequenced antibody.

In most embodiments, both the light and heavy chains from the same human antibody may be used as donors.

Various antibody databases can be searched to identify similar human antibody immunoglobulins (usually gemiline antibody sequences) for a given rabbit immunoglobulin sequence. In addition to National Center for Biotechnology Information (NCBI) databases, several of the most commonly used databases are listed below:

V BASE—Database of Human Antibody Genes: This database is maintained by the medical research council (MRC), of Cambridge UK and is provided via the world wide website at mrc-cpe.cam.ac.uk. This database is comprehensive directory of all human germline variable region sequences compiled from over a thousand published sequences, including those in the current releases of the Genbank and EMBL data libraries.

Kabat Database of Sequences of Proteins of Immunological Interest (Johnson, G and Wu, T T (2001) Kabat Database and its applications: future directions. Nucleic Acids Research, 29: 205-206) found at the website of Northwestern University, Chicago (immuno.bme.nwu.edu). The kabat database is also available at the nih/ncbi site

Immunogenetics Database: Maintained by and found at the website of the European Bioinformatics Institute: www.ebi.ac.uk. This database is integrated specialized database containing nucleotide sequence information of genes important in the function of the immune system. It collects and annotates sequences belonging to the immunnglobulin superfamily which are involved in immune recognition.

ABG: Germline gene directories of the mouse—a directory of mouse VH and VK germline segments, part of the webpage of the Antibody Group at the Instituto de Biotecnologia, UNAM (National University of Mexico).

Built-in searching engines can be used to search for similar sequences in terms of amino acid sequence homology. In the methods of this invention, BLAST (Altschul et al., J. Mol. Biol. 215:403-10, 1990) is performed using default parameters, including choosing the BLOSUM62 matrix, an expect threshold of 10, low complexity filter off, gaps allowed, and a word size of 3.

Humanizing a Rabbit Monoclonal Antibody

The subject invention provides methods by which a rabbit antibody may be humanized. In the subject methods, the framework regions of a rabbit antibody VH and VL domains may be modified so that they become more similar to the similar human antibody identified above. In general, these methods are generally compatible (i.e., may be performed in addition to) other humanization methods (e.g., CDR grafting, antibody resurfacing, etc.) to humanize a rabbit antibody.

In general, the methods involve aligning the sequence of the VH and VL domains of the parental antibody with the VH and VL domains of the donor antibody, and altering the sequences of the VH and VL framework domains of the parental antibody to make it more like the sequence of the donor antibody. In general, this involves substituting particular amino acids in the rabbit antibody sequence with corresponding (i.e., at the same position, according to the number schemes described above) amino acids of the donor antibody. In other words, “corresponding” means an amino acid residue on a donor sequence is positioned across from a residue on a parent sequence when the two sequences are aligned. Of course, as is known in the art (e.g. Roguska et al, P.N.A.S. 91: 969-973, 1994; Kabat 1991 Sequences of Proteins of Immunological Interest, DHHS, Washington, D.C.), sometimes one, two or three gaps and/or insertions of up to one, two, three or four or more amino acids should be made to one or both of the sequences in order to accomplish an alignment. As such, in many embodiments, spaces are inserted into or amino acids are deleted from a parent rabbit antibody sequence in order to accomplish an alignment between the parent rabbit sequence and the human sequence.

In other embodiments, the subject methods involves substituting a region of the rabbit antibody with a region from the donor antibody. A substituted region may add or remove amino acids, as compared to the parent antibody sequence. In most embodiments the substituted amino acids are not contiguous, and may consist of a group of non-contiguous amino acids that are different between the parental or donor antibody. Accordingly, in some embodiments, the methods involve substituting the framework regions of a donor human antibody into the framework regions of a parental human antibody, according to the limitations discussed below, to humanize the rabbit antibody. If an additional amino acid is present in the human antibody sequence, as compared to the rabbit antibody sequence, it is usually added to the rabbit antibody sequence, and, similarly, if an amino acid is absent in the human antibody sequence, as compared to the rabbit antibody sequence, it is usually removed from the rabbit antibody sequence during humanization.

Variable Domain N-termini:

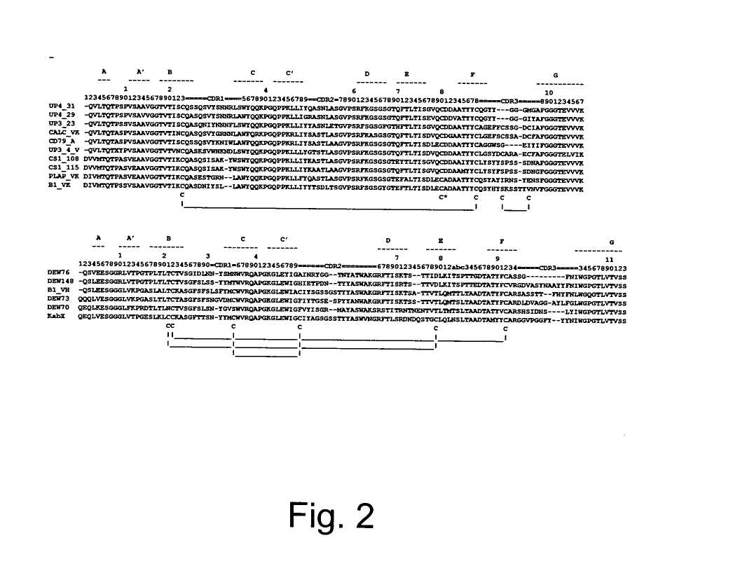

The VH domains of antibodies from all three major VH1 allotypes (A1, A2, A3) from rabbit have predicted N-termini (i.e., the FR1 domains of these antibodies) that are short by one residue, as compared to the N-termini of human VH chains. However, because VH genes other than VH1 genes may be utilized in rabbits less frequently than VH1 genes, not all rabbit antibody heavy chains have an N-terminus that is shorter. In practice, approximately half of the variable kappa chains that we have cloned are short by one residue at their N-termini relative to other rabbit VKs and to all human VKs (see FIG. 2).

In general, with the exception of certain amino acids noted below, the entire FR1 domain, (i.e., the N-terminal domain of the heavy chain of an antibody that is N-terminal to the first amino acid of the first CDR region (CDR1), including the A, and A′ strands and part of the B strand) of the rabbit antibody is replaced with the entire FR1 domains of the donor antibody such that the first three N-terminal residues (1, 2 and 3) of the humanized rabbit antibody heavy and light chains matches perfectly with the first three N-terminal residues of the donor human antibody heavy and light chains. In most embodiments of the invention, residues VK22, VH24, VH27, VH28, VH29 and VH30 should not be changed because they are in close proximity of the CDRs. Conservative amino acid substitutions of residues VK11, VK13, VK19, VH9, VH12 and VH18 can be made.

VHD-E Loops:

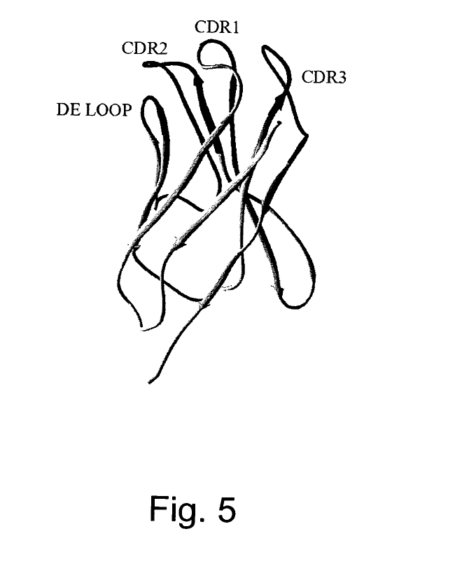

FIG. 5 shows the position of the D-E loop with respect to the CDRs of an antibody heavy chain. Two of the three major VH1 allotypes (A1, A3) have D-E loop domains that are frequently short by two residues or by one residue respectively, as compared to human and rabbit A2 allotype VH chains. During affinity maturation the number of amino acid residues in this region can change but usually it does not. According to this invention, the D-E loop is humanized by replacing six adjacent rabbit residues, positions 72 through 77, with corresponding residues found in the selected donor antibody sequence. In some embodiments, the following sequences may be used to substitute residues 72-77 of the rabbit sequence: DTSKNQ (SEQ ID NO:24), DNSKNT (SEQ ID NO:25), DNAKNS (SEQ ID NO:26), or, in some embodiments: DDSKNS (SEQ ID NO:27), DDSKNT (SEQ ID NO:28), DESTST (SEQ ID NO:29), DGSKSI (SEQ ID NO:30), DKSIST (SEQ ID NO:31), DKSKNQ (SEQ ID NO:32), DKSTST (SEQ ID NO:33), DMSTST (SEQ ID NO:34), DNAKNT (SEQ ID NO:35), DNSKNS (SEQ ID NO:36), DRSKNQ (SEQ ID NO:37), DRSMST (SEQ ID NO:38), DTSAST (SEQ ID NO:39), DTSIST (SEQ ID NO:40), DTSKSQ (SEQ ID NO:41), DTSTDT (SEQ ID NO:42), DTSTST (SEQ ID NO:43), DTSVST (SEQ ID NO:44), ENAKNS (SEQ ID NO:45), or NTSIST (SEQ ID NO:46) may be used.

In alternative embodiments, rabbit antibodies having the correct length of D-E loop may be obtained from rabbits that are homozygous for A2.

VK C80/E-F Loops:

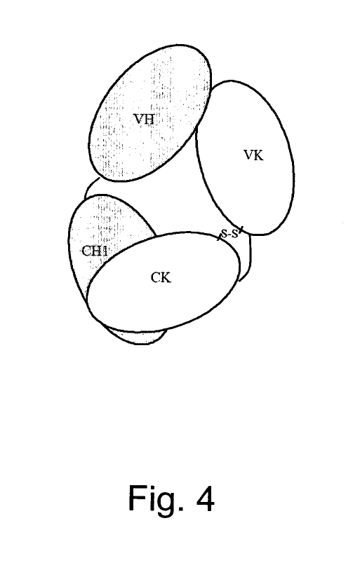

Rabbit antibody kappa chains, such as those belonging to the kappa-1 b4, b5, and b6 allotypes, have a cysteine residue at position 80 (cys80) that forms a disulfide bond with a cys residue in the kappa constant domain (see FIG. 4). If present in a rabbit antibody, cys80 should be mutated to a non-cysteine residue. In general, while cys80 may be substituted with any other amino acid, pro, ala or ser (P,A,S) are most usually used. In other embodiments a residue at the corresponding position (i.e. VK80) of the selected donor antibody may be used.

In alternative embodiments, rabbit antibodies that do not contain a cys at position 80 may be produced by using rabbits in which kappa chains lacking the VK-CK disulfide bond involving cys80 are produced. In particular, basilea (bas) rabbits make only VK kappa-2 isotypes and lambda chains, both of which don't have the subject disulfide bond. Also, antibodies from b9/b9 homozygous rabbits may be used since they do not don't utilize Cys 80. In antibodies from b9/b9 rabbits, cys 108 forms a disulphide bond instead.

In an alternative embodiment for replacing cys80 of a rabbit antibody, if present, the E-F loop (residues VK77 through VK83) of the parent rabbit antibody light chain is replaced by other sequences, such as those from the selected donor antibody. These 7 amino acids are usually replaced with the following amino acid sequences: SLQPEDF (SEQ ID NO:47) or RVEAEDV (SEQ ID NO:48); or NIESEDA (SEQ ID NO:49), RLEPEDF (SEQ ID NO:50), SLEAEDA (SEQ ID NO:51), SLEPEDF (SEQ ID NO:52), SLQAEDV (SEQ ID NO:53), SLQPDDF (SEQ ID NO:54), SLQPEDI (SEQ ID NO:55), SLQPEDV (SEQ ID NO:56) or SLQSEDF (SEQ ID NO:57). In certain embodiments, any corresponding sequence that is found in any human antibody, including that of the selected donor antibody, that does not have a cysteine may be used.

Among these 7 residues, the residue at position 82 must be always a D (asp). The residues at positions 78 and 83 should be left as found in the rabbit if the corresponding human residue is significantly different in size, electric charge or hydrophobicity because these residues are often buried. In most cases the rabbit residue at position 78 will be conserved (V, L, I, or M) in both rabbit and human VKs but the same is not true for residue 83 which can differ considerably in charge and size between rabbit and human VKs. Thus, in many embodiments, residue 83 is left intact while all EF loop amino acids can be changed to according to one of the sequences described above.

Other Cysteine Pairs:

For rabbit kappa chains that have a cysteine residue at position 108 such as those antibodies of the kappa-1 b9 allotype, that cysteine residue can be changed to any other residue, usually to a residue found at the same position in a human antibody, such as the selected donor antibody.

In addition to the VK cys80 or cys108, rabbit antibody variable regions often have other cysteines that are not present in human antibodies. By modeling, or comparison to a known structure, other cys pairs of the antibody that are in close proximity, i.e. close enough to bond via a disulphide bond, should be changed. In some embodiments a single cys of a pair of bonding cys residues is changed, whereas in other embodiments, both cys residues of the bonding pair are changed. In many embodiments, therefore, the subject procedure involves identifying a pair of cysteines that lie in close proximity to each other (e.g., within about 4, 5, 6, or about 7 Angstroms), and changing both of the cysteines to other amino acids. These cys residues may be changed to any other amino acid, usually a non-cysteine amino acid that is at a position corresponding to that of another antibody, such as the selected donor antibody.

In specific embodiments, the rabbit VH cysteine pair cys21/cys79 may be changed to: S21/Y79, T21/S79, or, in other embodiments, S21/H79 and T21/V79.

In general, putative cysteine pairs that are contained within one of the CDRs should not be changed. However, certain exceptions do exist. One exception are the VH35-VH50 cysteines, which are in CDRs (as defined by Kabat 1991, supra). According to structural models, the side chains of both of these cysteines are buried and moreover both cysteines occupy positions in a beta strand. Therefore, in this case the cysteines could be optionally changed to corresponding human residues.

The antibody modifications described above, when performed alone or in combination with any other method, should be done without modifying the amino acids set forth in Table 1, or, in other embodiments, any amino acid that is buried may be conservatively changed. These amino acids are further described below, and predicted to be amino acids that are in close proximity to a CDR, or a different chain, or are buried.

CDR contacts CDR H3 cannot usually be modeled with confidence regardless of the animal species that produced the antibody. In particular, a rabbit antibody containing a CDR (e.g., CDR L3) that is longer (e.g., 2, 3, 4, 5, 6, or 7 or more amino acids longer) than that of humans or mice is difficult model. As such, rabbit CDRs frequently cannot be readily assigned to known canonical structures on the basis of the protein sequence alone. Nevertheless, according to this invention one can still predict most framework residues that are likely to in close proximity with the CDRs because as CDR's get longer such as can be the case for CDR H3 and CDR L3, or as they assume different conformations they are more likely to change in loop regions that contact only the antigen or other CDRs but not framework residues. Therefore, even a rough model of a rabbit antibody is sufficient to predict CDR-contacting residues.

Interchain Contacts.

Many of the amino acids listed in Table 1 are involved in interchain contacts (e.g., at the VK/VH interface), and, as such, they should not be altered during humanization.

Buried Residues.

Buried residues (i.e., amino acids that are predicted to be not at the surface of an antibody) should not be changed during humanization, or, in some embodiments, may be substituted with amino acids of similar size and hydrophobicity to make conservative changes in the amino acid sequence (Table 1).

In many embodiments, up to 2, up to about 4, up to about 6, up to about 8, up to about 10, up to about 12, up to about 14, up to about 16 or up to about 20 amino acids are modified in each variable domain.

TABLE 1
Framework residues that may be structurally important because they
make CDR contacts (CDR) or interface contacts (INT), or because
they are buried (BUR).

VK
CDR: 1, 2, 3, 4, 5, 7, 22, 23, 35, 45, 48, 49, 58, 60, 62, 66, 67, 69, 70,
71, 88
TNT: 36, 38, 43, 44, 46, 85, 87, 98, 100
BUR: 6, 11, 13, 19, 21, 37, 47, 61, 73, 75, 78, 82, 83, 84, 86, 102, 104,
106
VH
CDR: 1, 2, 4, 24, 27, 28, 29, 30, 36, 38, 40, 46, 48, 49, 66, 67, 68, 69, 71,
73, 78, 80, 82, 86, 92, 93, 94,
INT: 37, 39, 43, 44, 45, 47, 91, 103, 105
BUR: 6, 9, 12, 18, 20, 22, 76, 82c, 88, 90, 107, 109, 111
Note: residues that belong to more than one class were listed only in one class (amino acids were listen in INT > CDR > BUR).

In many embodiments the subject methods are performed by an algorithm by a computer or a computer system. In these embodiments, a user inputs at least the amino acid sequence of a framework region or a variable domain of a rabbit antibody into a computer using, e.g., a user interface, the computer performs the methods as described above, and outputs a humanized rabbit framework or modified variable domain amino acid sequence or even a nucleotide sequence encoding a modified rabbit framework or modified variable domain a using an algorithm. Such programming is well within the abilities of one of skill in the art.

Programming according to the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture that includes a recording of the present programming/algorithms for carrying out the above described methodology.

HUMANIZED RABBIT MONOCLONAL ANTIBODIES

The present invention provides rabbit antibodies that are humanized by the method set forth above.

In general, a humanized rabbit antibody retains specificity for an antigen as compared to a parent antibody, has substantial affinity (e.g. at least 10⁷M⁻¹, at least 10⁸ M⁻¹, or at least 10⁹ M⁻¹ to 10¹⁰ M⁻¹ or more), and is usually less immunogenic in a human host, as compared to a parent rabbit antibody. In many embodiments, the modified rabbit antibody contains at least one set of contiguous or non-contiguous amino acids from a human antibody, as set forth above.

The level of immunogenicity of a humanized rabbit antibody as compared to a parent rabbit antibody in a human host may be determined by any of a number of means, including administering to a single human host a formulation containing equimolar amounts of the two isolated antibodies and measuring the immune response of the human host relative to each of the antibodies. Alternatively, the parent and modified antibodies are administered separately to different human hosts and the immune response of the hosts are measured. One suitable method for measuring the immune response of the non-rabbit host relative to each of the antibodies is by ELISA (described in Ausubel, et al, Short Protocols in Molecular Biology, 3rd ed., Wiley & Sons, 1995, UNIT 11-4), where a suitable equal amount of each antibody is spotted into the wells of a microtitre plate, and the assay is performed polyclonal antiserum from the human host. In most embodiments, a subject humanized rabbit antibody is about 10% less immunogenic, about 20% less immunogenic, about 30% less immunogenic, about 40% less immunogenic, about 50% less immunogenic, about 60% less immunogenic, about 80% less immunogenic, about 90% less immunogenic or even about 95% less immunogenic than an unmodified parent rabbit antibody.

Depending on the constant regions and other regions used, several types of antibody that are known in the art may be made by this method. As well as full length antibodies, antigen-binding fragments of antibodies may be made by the subject methods. These fragments include, but are not limited to, Fab, Fab′ and F(ab′)₂, Fd, single-chain Fvs (scFv), single-chain immunoglobulins (e.g., wherein a heavy chain, or portion thereof, and light chain, or portion thereof, are fused), disulfide-linked Fvs (sdFv), diabodies, triabodies, tetrabodies, scFv minibodies, Fab minibodies, and dimeric scFv and any other fragments comprising a V_{L} and a V_{H} domain in a conformation such that a specific antigen binding region is formed. Antibody fragments, including single-chain antibodies, may comprise the variable region(s) alone or in combination with the entire or partial of the following: a heavy chain constant domain, or portion thereof, e.g., a CH1, CH2, CH3, transmembrane, and/or cytoplasmic domain, on the heavy chain, and a light chain constant domain, e.g., a Cₖₐₚₚₐ or C_{lambda} domain, or portion thereof on the light chain. Also included in the invention are any combinations of variable region(s) and CH1, CH2, CH3, Cₖₐₚₚₐ, C_{lambda}, transmembrane and cytoplasmic domains. By the term “antibody” is meant any type of antibody, including those listed above, in which the heavy and light chains have been, as explained above, naturally paired, i.e., excluding so-called “phage-display” antibodies.

A humanized rabbit antibody may, of course, accommodate a level of amino acid variation, e.g. conservative amino acids substitutions, as long as they retain specificity, have substantial affinity and are usually less immunogenic in a non-rabbit host, as compared to a parent antibody.

NUCLEIC ACIDS ENCODING RABBIT MONOCLONAL ANTIBODIES

The invention further provides nucleic acids comprising a nucleotide sequence encoding a subject modified rabbit antibody, as well as portions thereof, including a light or heavy chain, a light or heavy chain variable domain, or a framework region of a light or heavy chain variable domain. Subject nucleic acids are produced by a subject method. In many embodiments, the nucleic acid also comprises a coding sequence for a constant domain, such as a constant domain of any human antibody. Nucleic acids encoding a human immunoglobulin leader peptide (e.g. MEFGLSWVFLVAILKGVQC, SEQ ID NO:58) may be engineered to allow the secretion of the antibody chains.

Since the genetic code and recombinant techniques for manipulating nucleic acid are known, and the amino acid sequences of the subject antibodies may be obtained using the method described above, the design and production of nucleic acids encoding a humanized rabbit antibody is well within the skill of an artisan. In certain embodiments, standard recombinant DNA technology (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.) methods are used. For example, antibody coding sequences may be isolated from antibody-producing cells using any one or a combination of a variety of recombinant methods that do not need to be described herein. Subsequent substitution, deletion, and/or addition of nucleotides in the nucleic acid sequence encoding a protein may also be done use standard recombinant DNA techniques.

For example, site directed mutagenesis and subcloning may be used to introduce/delete/substitute nucleic acid residues in a polynucleotide encoding an antibody. In other embodiments, PCR may be used. Nucleic acids encoding a polypeptide of interest may also be made by chemical synthesis entirely from oligonucleotides (e.g., Cello et al., Science (2002) 297:1016-8).

In certain embodiments, the codons of the nucleic acids encoding polypeptides of interest are optimized for expression in cells of a particular species, particularly a mammalian, e.g., human, species.

The invention further provides vectors (also referred to as “constructs”) comprising a subject nucleic acid. In many embodiments of the invention, the subject nucleic acid sequences will be expressed in a host after the sequences have been operably linked to an expression control sequence, including, e.g. a promoter. The subject nucleic acids are also typically placed in an expression vector that can replicate in a host cell either as an episome or as an integral part of the host chromosomal DNA. Commonly, expression vectors will contain selection markers, e.g., tetracycline or neomycin, to permit detection of those cells transformed with the desired DNA sequences (see, e.g., U.S. Pat. No. 4,704,362, which is incorporated herein by reference). Vectors, including single and dual expression cassette vectors are well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.). Suitable vectors include viral vectors, plasmids, cosmids, artificial chromosomes (human artificial chromosomes, bacterial artificial chromosomes, yeast artificial chromosomes, etc.), mini-chromosomes, and the like. Retroviral, adenoviral and adeno-associated viral vectors may be used.

A variety of expression vectors are available to those in the art for purposes of producing a polypeptide of interest in a cell. One suitable vector is pCMV, which used in certain embodiments. This vector was deposited with the American Type Culture Collection (ATCC) on Oct. 13, 1998 (10801 University Blvd., Manassas, Va. 20110-2209 USA) under the provisions of the Budapest Treaty for the International Recognition of the Deposit of Microorganisms for the Purpose of Patent Procedure. The DNA was tested by the ATCC and determined to be viable. The ATCC has assigned the following deposit number to pCMV: ATCC #203351.

The subject nucleic acids usually comprise an single open reading frame encoding a subject antibody, however, in certain embodiments, since the host cell for expression of the polypeptide of interest may be a eukaryotic cell, e.g., a mammalian cell, such as a human cell, the open reading frame may be interrupted by introns. Subject nucleic acid are typically part of a transcriptional unit which may contain, in addition to the subject nucleic acid 3′ and 5′ untranslated regions (UTRs) which may direct RNA stability, translational efficiency, etc. The subject nucleic acid may also be part of an expression cassette which contains, in addition to the subject nucleic acid a promoter, which directs the transcription and expression of a polypeptide of interest, and a transcriptional terminator.

Eukaryotic promoters can be any promoter that is functional in a eukaryotic, or any other, host cell, including viral promoters and promoters derived from eukaryotic or prokaryotic genes. Exemplary eukaryotic promoters include, but are not limited to, the following: the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gall gene sequence promoter (Johnston et al., Proc. Natl. Acad. Sci. (USA) 79:6971-6975, 1982); Silver et al., Proc. Natl. Acad. Sci. (USA) 81:5951-59SS, 1984), the CMV promoter, the EF-1 promoter, Ecdysone-responsive promoter(s), tetracycline-responsive promoter, and the like. Viral promoters may be of particular interest as they are generally particularly strong promoters. In certain embodiments, a promoter is used that is a promoter of the target pathogen. Promoters for use in the present invention are selected such that they are functional in the cell type (and/or animal) into which they are being introduced. In certain embodiments, the promoter is a CMV promoter.

In certain embodiments, a subject vector may also provide for expression of a selectable marker. Suitable vectors and selectable markers are well known in the art and discussed in Ausubel, et al, (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.). A variety of different genes have been employed as selectable markers, and the particular gene employed in the subject vectors as a selectable marker is chosen primarily as a matter of convenience. Known selectable marker genes include: the thimydine kinase gene, the dihydrofolate reductase gene, the xanthine-guanine phosporibosyl transferase gene, CAD, the adenosine deaminase gene, the asparagine synthetase gene, the antibiotic resistance genes, e.g. tetr, ampr, Cmr or cat, kanr or neor (aminoglycoside phosphotransferase genes), the hygromycin B phosphotransferase gene, and the like.

The subject nucleic acids may also contain restriction sites, multiple cloning sites, primer binding sites, ligatable ends, recombination sites etc., usually in order to facilitate the construction of a nucleic acid encoding a humanized rabbit antibody.

In general, several methods are known in the art for producing antibody-encoding nucleic acids, including those found in U.S. Pat. Nos. 6,180,370, 5,693,762, 4,816,397, 5,693,761 and 5,530,101. One PCR method utilizes “overlapping extension PCR” (Hayashi et al., Biotechniques. 1994: 312, 314-5) to create expression cassettes for the heavy and light chain encoding nucleic acids. In this method multiple overlapping PCR reactions using the cDNA product obtained from the antibody producing cell and other appropriate nucleic acids as templates generates an expression cassette.

METHODS FOR PRODUCING HUMANIZED RABBIT MONOCLONAL ANTIBODIES

In most embodiments, the subject nucleic acids encoding a humanized monoclonal antibody are introduced directly into a host cell, and the cell incubated under conditions sufficient to induce expression of the encoded antibody.

Any cell suitable for expression of expression cassettes may be used as a host cell. For example, yeast, insect, plant, etc., cells. In many embodiments, a mammalian host cell line that does not ordinarily produce antibodies is used, examples of which are as follows: monkey kidney cells (COS cells), monkey kidney CVI cells transformed by SV40 (COS-7, ATCC CRL 165 1); human embryonic kidney cells (HEK-293, Graham et al. J. Gen Virol. 36:59 (1977)); baby hamster kidney cells (BHK, ATCC CCL 10); chinese hamster ovary-cells (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TRI cells (Mather et al., Annals N. Y. Acad. Sci 383:44-68 (1982)); NIH/3T3 cells (ATCC CRL-1658); and mouse L cells (ATCC CCL-1). Additional cell lines will become apparent to those of ordinary skill in the art. A wide variety of cell lines are available from the American Type Culture Collection, 10801 University Boulevard, Manassas, Va. 20110-2209.

Methods of introducing nucleic acids into cells are well known in the art. Suitable methods include electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995. In some embodiments lipofectamine and calcium mediated gene transfer technologies are used.

After the subject nucleic acids have been introduced into a cell, the cell is typically incubated, normally at 37° C., sometimes under selection, for a period of about 1-24 hours in order to allow for the expression of the antibody. In most embodiment, the antibody is typically secreted into the supernatant of the media in which the cell is growing in.

In mammalian host cells, a number of viral-based expression systems may be utilized to express a subject antibody. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). The efficiency of expression maybe enhanced by the inclusion of appropriate transcription enhancer elements, transcription terminators, etc. (see Bittner et al., Methods in Enzymol. 153:51-544 (1987)).

For long-term, high-yield production of recombinant antibodies, stable expression may be used. For example, cell lines, which stably express the antibody molecule may be engineered. Rather than using expression vectors which contain viral origins of replication, host cells can be transformed with immunoglobulin expression cassettes and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into a chromosome and grow to form foci which in turn can be cloned and expanded into cell lines. Such engineered cell lines may be particularly useful in screening and evaluation of compounds that interact directly or indirectly with the antibody molecule.

Once an antibody molecule of the invention has been produced, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In many embodiments, antibodies are secreted from the cell into culture medium and harvested from the culture medium.

DETERMINING BINDING AFFINITY OF HUMANIZED RABBIT ANTIBODIES

Once a modified antibody is produced, it is usually tested for affinity using any known method, such as 1) competitive binding analysis using labeled (radiolabeled or fluorescent labeled) parent rabbit antibody, the modified antibody and an antigen recognized by the parent antibody; 2) surface plasmon resonance using e.g. BIACore instrumentation to provide the binding characteristics of an antibody. Using this method antigens are immobilized on solid phase chips and the binding of antibodies in liquid phase are measured in a real-time manner; and 3) flow cytometry, for example, by using fluorescent activated cell sorting (FACS) analysis to study antibody binding to cell surface antigens; 4) ELISA; 5) equibrilium dialysis, or FACS. In this FACS method both transfected cells and native cells expressing the antigen can be used to study antibody binding. Methods for measuring binding affinity are generally described in Harlow et al,. Antibodies: A Laboratory Manual, First Edition (1988) Cold spring Harbor, N.Y.; Ausubel, et al, Short Protocols in Molecular Biology, 3rd ed., Wiley & Sons, 1995).

If affinity analysis reveals a decrease in antibody binding for the modified antibody as compared to its parent antibody, framework “fine tuning” may be performed to increase the affinity. One method of doing this is to systematically change back each modified residues by site-directed mutagenesis. By expressing and analyzing these back mutant antibodies, one would predict the key residues that cannot be modified unless without decreasing affinity.

An alternative method to predict the residues that may need back-mutation is through molecular modeling. By comparing the 3-dimensional models of original and humanized or murinized antibody structure, any residues from the surface residues that are too close (e.g. less than about 5 Angstroms) to the CDR residues, should be back-mutated to a residue of the rabbit or to a common residue for both species.

UTILITY

A humanized rabbit antibody of the present invention find use in diagnostics, in antibody imaging, and in treating diseases susceptible to monoclonal antibody-based therapy. In particular, a humanized rabbit antibody may be used for passive immunization or the removal of unwanted cells or antigens, such as by complement mediated lysis or antibody mediated cytotoxicity (ADCC), all without substantial immune reactions (e.g., anaphylactic shock) associated with many prior antibodies. For example, the antibodies of the present invention may be used as a treatment for a disease where the surface of an unwanted cell specifically expresses a protein recognized the antibody (e.g. HER2) or the antibodies may be used to neutralize an undesirable toxin, irritant or pathogen. Humanized rabbit immunoglobulins are particularly useful for the treatment of many types of cancer, for example colon cancer, lung cancer, breast cancer prostate cancer, etc., where the cancers are associated with expression of a particular cellular marker. Since most, if not all, disease-related cells and pathogens have molecular markers that are potential targets for antibodies, many diseases are potential indications for humanized antibody drug. These include autoimmune diseases where a particular type of immune cells attack self-antigens, such as insulin-dependent diabetes mellitus, systemic lupus erythematosus, pernicious anemia, allergy and rheumatoid arthritis; transplantation related immune activation, such as graft rejection and graft-vs-host disease; other immune system diseases such as septic shock; infectious diseases, such as viral infection or bacteria infection; cardiovascular diseases such as thrombosis and neurological diseases such as Alzeimer's disease.

KITS

Also provided by the subject invention are kits for practicing the subject methods, as described above. The subject kits at least include one or more of: a nucleic acid encoding at least one framework sequence of a humanized rabbit antibody, as set forth above, a vector containing the same, oligonucleotides primers for amplifying the same. Other optional components of the kit include: restriction enzymes, control primers and plasmids; buffers; etc. The nucleic acids of the kit may also have restrictions sites, multiple cloning sites, primer sites, etc to facilitate their ligation to non-rabbit antibody CDR-encoding nucleic acids. The various components of the kit may be present in separate containers or certain compatible components may be precombined into a single container, as desired.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Also provided by the subject invention is are kits including at least a computer readable medium including programming as discussed above and instructions. The instructions may include installation or setup directions. The instructions may include directions for use of the invention with options or combinations of options as described above. In certain embodiments, the instructions include both types of information.

Providing the software and instructions as a kit may serve a number of purposes. The combination may be packaged and purchased as a means for producing rabbit antibodies that are less immunogenic in a non-rabbit host than a parent antibody, or nucleotide sequences them.

The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc, including the same medium on which the program is presented.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Rabbit Monoclonal Antibodies

FIG. 2 depicts sequence alignments of variable heavy and kappa chains cloned from various rabbit monoclonal developed at Epitomics. It demonstrates structural features of rabbit chains that are unusual relative to those of human and murine antibodies. A majority of the VK chains and half of the VH chains are short by one residue on the N-terminus (relative to other rabbit sequences as to human sequences). A majority of the heavy chains is also short by one or two residues in the D-E loop region. All isolated kappa chains have a cysteine residue at position 80. Many of the kappa chain CDR3 sequences are longer than those of any previously known human or murine antibody. A few kappa chains have an extra pair of cysteine residues in their third CDR. Additional pairs of cysteines are also present in some of the VH regions. Finally, while this fact is not clearly demonstrated in the figure, some of the CDRs cannot be assigned to a previously known canonical structure.

### Example 2

### Humanization of Rabbit Monoclonal Antibody B1

The variable kappa and heavy chains of the rabbit anti-integrin beta-6 monoclonal antibody B1 were PCR-cloned as follows. Several independent PCR products were sequenced and one PCR reaction with one set of primers is usually sufficient.

```
Preparation of a hybridoma cell suspension
spin 1 ml growing B1 cells 1100 RPM 5 min
wash with 1X PBS
count cells and adjust to 400,000 cells/ml
Preparation of RNA
Add 1 ul cells to 9 ul Buffer A on ice
Add 5 ul cold Buffer B
heat to 65° C. 1 mm
cool gradually in Thermocycler
55° C.  45° C.  35° C.  23° C.  Ice
30 sec  30 sec  30 sec  2 min
Add cold Buffer C - 5 ul per tube
Incubate at 42° C. for 42 min
put back in Ice
BUFFERS A, B, C
Buffer A
2 ul DTT (0.1 M)
2 ul 5X first strand buffer
5 ul DEPC treated H2O
Buufer B
1.0 ul 0.1% NP40
1.0 ul First strand buffer
1.0 ul oligo dT
0.5 ul RNAseOUT 40 U/ml
1.5 ul DEPC treated H2O
Buffer C
1 ul 10 mM dNTP mix
1 ul 5X First strand buffer (Invitrogen)
1 ul Superscript RTII (Invitrogen)
2 ul DEPC treated H2O
PCR
primer concentration: 3 pmole/ul
  2.50 ul 10x buffer
  0.75 ul 50 mM MgCl2
  3.00 ul primer 1
  3.00 ul primer 2
  0.50 ul 10 mM dNTP mix
  0.25 ul Taq or other polymerase
10.00 ul Water
  5.00 ul template
25.00 ul
94° C. 2 min
94° C. 30 sec|
57° C. 30 sec| × 40 cycles
68° C. 25 sec|
68° C. 10 min
First round: use for H chain: Primer 1 + Primer 10
                                  for L chain : Primer 12 +
                                  Primer 19
Nested PCR for H chain only: Primer 2 +
                                  Primer 8
```

| | |
|---|---|
| >PRIMER 1 TCGCACTCAACACAGACGCTCACC | (SEQ ID NO:59) |
| >PRIMER 2 ATGGAGACTGGGCTGCGCTGGCTT | (SEQ ID NO:60) |
| >PRIMER 8 GCTCAGCGAGTAGAGGCCTGAGGAC | (SEQ ID NO:61) |
| >PRIMER 10 TTGGGGGGAAGATGAAGACAGACGG | (SEQ ID NO:62) |
| >PRIMER 12 CAGTGCAGGCAGGACCCAGCATGG | (SEQ ID NO:63) |
| >PRIMER 19 GCCCTGGCAGGCGTCTCRCTCTA | (SEQ ID NO:64) |

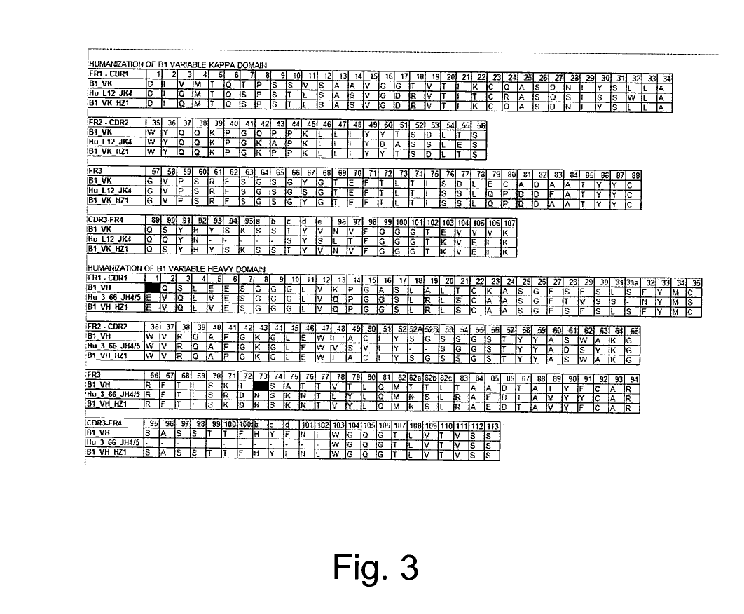

CDRs and residue numbers were assigned as to match the numbering described in Chothia 1998, supra and Kaba supra t. FIG. 3 summarizes the sequence-planning for the humanization of the anti-integrin beta-6 rabbit monoclonal antibody B1. The details will follow in the sections below. Overall 15 and 17 framework residues were mutated from rabbit to human identities in VK and VH, respectively. Two residues were inserted in VH at positions 1 and 73 respectively. Four and seven framework residues were left unchanged, in VK and VH, respectively, relative to the maximum number of changes that could be made. The percent ID in the VK and VH frameworks increased from 76% to 95% and from 72% to 94%, respectively.

Many of the following humanization steps require a detailed knowledge of antibody variable region structure. The most reliable and easiest way to acquire such knowledge is from the extensive antibody structure literature (see for example Chothia 1998, supra). Nevertheless, is it may be useful to visualize a few of the hundreds of antibody structures publicly available at the pdb database as well as that of the particular rabbit antibody that is to be humanized.

In order to model the rabbit antibody the rabbit sequences are blasted against the pdb database to find a suitable structure for performing homology modeling. One can usually find structures of paired VH/VL chains whose protein sequences are very similar to those of the rabbit antibody. Naturally, the closest the similarities between sequences the better the resulting model will be.

There are several programs that can be used to build a model by homology. Some of these programs can be purchased but some are also available through the internet. For example, the Swiss Pdb Viewer, also known as “Deep View” can be used to model proteins by homology. If there are gaps or insertions in loops of the rabbit antibody relative to the loops of the template structure, those can be modeled using other structures. CDRs may be easy to model if they belong to a known canonical structure. This will almost certainly be always true for CDR L2, for example. However, it is frequently not possible to assign canonical structures to rabbit CDRs and it may be difficult to find good template structures to model them. In particular, one can expect great difficulty in finding good structural templates for CDRs L3 and H3. It may be difficult to find a good model for the D-E loop as well. Neverthless, the modeling of the CDR loops and of the D-E loop does not have to be perfect.

The program pdb viewer can be used to determine which framework residues are likely to make contacts with the CDRs. Alternatively, one can write a script in a number of programming languages such as PERL, or even use Microsoft Excel, that can take the coordinates from the pdb file directly and determine which residues are within a contact distance of 5 angstroms or less from any CDR residue. Because the rabbit antibody model is not expected to be perfect it is advisable to be conservative and actually calculate residues that are at 6 or even 7 angstroms from the CDRs and then visualize them in order to decide if they are likely to contact CDRs. The same approach can be used to find which residues are likely to be involved in interchain contacts although in this case it is better to examine several structures visually using pdb viewer. Finally, one can use the pdb viewer script language to calculate relative surface accessibilities and thus determine which residues are buried.

The B1 rabbit monoclonal antibody B1 has that predicted disulfide bond and therefore cannot be humanized unless the cysteine 80 is replaced by a non-cysteine human residue. According to this invention cys 80 and the adjacent residues from positions 77 through 83 were changed from DLECADA (SEQ ID NO:65) to SLQPDDA(SEQ ID NO:66). The humanized sequence is almost identical to one of the sequences set forth above, SLQPDDF(SEQ ID NO:67), except that the last residue at position 83 was not changed from A to F. This was because, also according to this invention that residue should be left intact in the side chain of the replacement is too different. This residue is often buried and in this case a change from A to F would mean that a large methyl-phenyl group would have to fit in where previously only a methyl group was found.

The N-termini of both chains were completely humanized up to residue 21 of VK and residue 27 of VH respectively. Residues VK22, VH 28 and VH29 were not changed because they are too close to or make contacts with the CDRs.

FIG. 5 depicts the modeled structure of a rabbit VH domain showing the positions of the three CDRs and the D-E loop. The later is close to the CDRs. This region of the rabbit monoclonal antibody B1 was humanized by adopting one of the three best possible human sequences: DNSKNT (positions 72-77) which includes an extra residue and therefore became a larger loop in the humanized versus the rabbit antibody.

The cys35-cys50 pair present in the VH of the rabbit B1 antibody was not changed, because both residues are predicted to be buried and both are part of CDRs.

All other residues were changed to match the human target sequence except for the following:
VK43 and VH 91 because they were close to or in at the VK/VH interface.
VK83 because it is likely to be buried and the change (A to F) would alter, its size considerably.
VK67, VH48, VH49, VH71, and VH78 because they are close to or contact the CDRs

In this case, the two planned variable regions of the B1 antibody were synthesized and cloned into expression vectors that encoded the constant regions of human kappa and IgG1. The vectors were then transiently expressed in HEK293 cells and culture supernatants were used in a cell ELISA experiment that demonstrated that the humanized antibody bound antigen strongly.

In other cases one might make point mutations instead of synthesizing the variable region genes. One might also express antibody fragments instead of whole IgG.

It is evident from the above results and discussion that the subject invention provides an important new means for humanizing a rabbit antibody. As such, the subject methods and systems find use in a variety of different applications, including research, agricultural, therapeutic and other applications. Accordingly, the present invention represents a significant contribution to the art.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of humanizing a rabbit monoclonal antibody, said method comprising:
(a) comparing an amino acid sequences of a heavy an a light chain variable domain of a parent rabbit antibody to the amino acid sequence of a heavy and a light chain variable domain of a similar human antibody; and
(b) altering amino acids within the framework regions of said heavy and light chain variable domains of said rabbit antibody such that the altered framework regions are more similar in sequence to the equivalent framework regions of said similar human antibody;
wherein said altered amino acids are not involved in complementarity determining region (CDR) contacts, interchain contacts, or are buried residues with substantially different side chains.

2. The method of claim 1, wherein said light chain of said parent monoclonal antibody contains a CDR 3 that is at least one amino acid longer than the CDR3 of said human antibody.

3. The method of claim 1, wherein the specificity and affinity said parent rabbit antibody and said altered rabbit antibody is substantially identical.

4. The method according to claim 1, wherein said altered rabbit antibody is less immunogenic in a human host than said rabbit parent antibody.

5. The method of claim 1, wherein the framework region 1 of said parent rabbit monoclonal antibody is replaced by a framework region 1 of said human antibody to extend the length of said parent rabbit monoclonal antibody by one or more amino acids.

6. The method of claim 1, wherein the VH D-E loop of said parent rabbit monoclonal antibody is replaced by a DE loop from said human antibody to extend the length of said D-E loop of said parent rabbit monoclonal by one or more amino acids.

7. The method of claim 1, wherein any VK cysteine residue at position 80 of said parent rabbit monoclonal antibody is replaced by an amino acid found at position 80 of said human antibody.

8. The method of claim 1, wherein the VK E-F loop of said parent rabbit monoclonal antibody is replaced by a E-F loop from said human antibody.

9. The method of claim 1, wherein pairs of cysteines that are situated in close proximity to each other in said parent monoclonal antibody are replaced by amino acids found at the same positions of said human antibody.

10. A rabbit monoclonal antibody humanized by the method set forth in claim 1.

11. The humanized rabbit monoclonal antibody of claim 13, wherein said antibody has a measured binding affinity of 2×10⁷ M⁻¹ or greater for an antigen that is bound with a binding affinity of 10⁸ M⁻¹ or greater by said parent rabbit antibody.

12. The modified rabbit antibody according to claim 10, wherein said antibody is not linked to a fragment of a viral polypeptide.

13. The modified rabbit antibody according to claim 10, wherein said antibody is an antibody fragment.

14. A nucleic acid encoding a heavy or light chain variable domain of the monoclonal antibody set forth in claim 10.

15. A vector comprising the nucleic acid of claim 14.

16. A host cell comprising the vector according to claim 15.

17. A method of producing a humanized rabbit antibody, said method comprising:
incubating the host cell of claim 16 under conditions sufficient to produce said antibody; and
harvesting said antibody.

18. A kit containing:
a monoclonal antibody humanized by the method of claim 1; and
instructions for using the monoclonal antibody to for treatment of a condition.

19. A computer-readable medium encoding instructions to direct a processor to perform the method of claim 1.

20. A kit for use in a computer, said kit comprising:
(a) a computer-readable medium according to claim 19; and
(b) instructions for operating said computer according to said programming.


SEQUENCE LISTING (67 sequences)

SEQ ID NO: 1 - protein, 109 aa, Artificial sequence
  Synthetic sequence
QVLTQTPSPVSAAVGGTVTISCQSSQSVYSNNRLSWYQQKPGQPPKLLIYQASNLASGVPSRFKGSGSGTQFTLTISGVQCDDAATYYCQGYYGGGMGAFGGGTEVVVK

SEQ ID NO: 2 - protein, 109 aa, Artificial sequence
  Synthetic sequence
QVLTQTPSPVSAVVGGTVTISCQASQSVYSNNRLAWYQQKPGQPPKLLIGRASNLASGVPSRFKGSGSGTQFTLTISEVQCDDVATYYCQGYYGGGIYAFGGGTEVVVK

SEQ ID NO: 3 - protein, 112 aa, Artificial sequence
  Synthetic sequence
QVLTQTPSSVSAAVGGTVTISCQASQNIYNNNFLSWYQQKPGQPPKLLIYYASNLETGVPSRFSGSGFGTHFTLTISGVQCDDAATYYCAGEFFCSSGDCIAFGGGTEVVVK

SEQ ID NO: 4 - protein, 112 aa, Artificial sequence
  Synthetic sequence
QVLTQTASPVSAAVGGTVTINCQASQSVYGRNNLAWFQRKPGQPPKRLIYSASTLASGVPSRFKASGSGTQFTLTISDVQCDGAATYYCLGEFSCSSADCFAFGGGTEVVVK

SEQ ID NO: 5 - protein, 109 aa, Artificial sequence
  Synthetic sequence
QVLTQTASPVSAAVGGTVTISCQSSQSVYKNIWLAWFQQKPGQPPKRLIYSASTLAAGVPSRFKGSGSGTQFTLTISDLECDDAATYYCAGGWSGEIYIFGGGTEVVVK

SEQ ID NO: 6 - protein, 112 aa, Artificial sequence
  Synthetic sequence
QVLTQTXYPVSAAVGGTVTVNCQASKSVWNKNDLSWYQQKPGQPPKLLLYGTSTLASGVPSRFKGSGSGTQFTLTISDVQCDDAATYYCLGSYDCARAECFAFGGGTELVIK

SEQ ID NO: 7 - protein, 112 aa, Artificial sequence
  Synthetic sequence
DVVMTQTPASVEAAVGGTVTIKCQASQSISAKYWSWYQQKPGQPPKLLIYKASTLASGVPSRFKGSGSGTEYTLTISGVQCDDAAIYYCLYSYYSPSSSDNAFGGGTEVVVK

SEQ ID NO: 8 - protein, 112 aa, Artificial sequence
  Synthetic sequence
DVVMTQTPASVEAAVGGTVTIKCQASQSISAKYWSWYQQKPGQPPKLLIYKAATLAAGVPSRFKGSGSGTQFTLTISGVQCDDAAMYYCLYSYFSPSSSDNGFGGGTEVVVK

SEQ ID NO: 9 - protein, 111 aa, Artificial sequence
  Synthetic sequence
DIVMTQTPASVEAAVGGTVTIKCQASESTGRNLAWYQQKPGQPPKLLFYQASTLASGVPSRFKGSGSGTEFALTISDLECADAATYYCQSYAYIRNSYENSFGGGTEVVVK

SEQ ID NO: 10 - protein, 112 aa, Artificial sequence
  Synthetic sequence
DIVMTQTPSSVSAAVGGTVTIKCQASDNIYSLLAWYQQKPGQPPKLLIYYTSDLTSGVPSRFSGSGYGTEFTLTISDLECADAATYYCQSYHYSKSSTYVNVFGGGTEVVVK

SEQ ID NO: 11 - protein, 110 aa, Artificial sequence
  Synthetic sequence
QSVEESGGRLVTPGTPLTLTCTVSGIDLNNYSMNWVRQAPGKGLEYIGAINRYGGTNYATWAKGRFTISKTSTTIDLKITSPTTGDTATYFCASSGFNIWGPGTLVTVSS

SEQ ID NO: 12 - protein, 119 aa, Artificial sequence
  Synthetic sequence
QSLEESGGRLVTPGTPLTLTCTVSGFSLSSYYMTWVRQAPGKGLEWIGHIETPDNTYYASWAKGRFTISRTSTTVDLKITSPTTEDTATYFCVRGDVASYNAAYYFNIWGPGTLVTVSS

SEQ ID NO: 13 - protein, 121 aa, Artificial sequence
  Synthetic sequence
QSLEESGGGLVKPGASLALTCKASGFSFSLSFYMCWVRQAPGKGLEWIACIYSGSSGSTYYASWAKGRFTISKTSATTVTLQMTTLTAADTATYFCARSASSTTFHYFNLWGQGTLVTVSS

SEQ ID NO: 14 - protein, 122 aa, Artificial sequence
  Synthetic sequence
QQQLVESGGGLVKPGASLTLTCTASGFSFSNGVDMCWVRQAPGKGLEWIGFIYTGSESPYYANWAKGRFTISKTSSTTVTLQMTSLTAADTATYFCARDLDVAGGAYLFGLWGPGTLVTVSS

SEQ ID NO: 15 - protein, 118 aa, Artificial sequence
  Synthetic sequence
QEQLKESGGGLFKPRDTLTLNCTVSGFSLSNYGVSWVRQAPGKGLEWIGFVYISGRMAYASWAKSRSTITRNTNENTVTLTMTSLTAADTATYVCARSHSIDNSLYIWGPGTLVTVSS

SEQ ID NO: 16 - protein, 122 aa, Artificial sequence
  Synthetic sequence
QEQLVESGGGLVTPGESLKLCCKASGFTTSNYYMCWVRQAPGKGLEWIGCIYAGSGSSTYYASWVNGRFTLSRDNDQSTGCLQLNSLTAADTAMYYCARGGVPGGFYYYNIWGPGTLVTVSS

SEQ ID NO: 17 - protein, 112 aa, Artificial sequence; identical to SEQ ID NO: 10
  Synthetic sequence

SEQ ID NO: 18 - protein, 63 aa, Artificial sequence
  Synthetic sequence
DIMTQPSSAVGVTICQWYQQKPGPKLLIYDGVPSRFSGSGGTEFTLTISLDATYYCFGGGTVK

SEQ ID NO: 19 - protein, 96 aa, Artificial sequence
  Synthetic sequence
DIMTQPSSAVGVTIKCQASDNIYSLLAWYQQKPGPPKLLIYYTSDLTSGVPSRFSGSGGTEFTLTISLDAATYYCQSYHYSKSSTYVNVFGGGTVK

SEQ ID NO: 20 - protein, 121 aa, Artificial sequence; identical to SEQ ID NO: 13
  Synthetic sequence

SEQ ID NO: 21 - protein, 61 aa, Artificial sequence
  Synthetic sequence
LESGGGLVPGSLLCASGFSWVRQAPGKGLEWRFTISSTLQMLADTAYCARWGQGTLVTVSS

SEQ ID NO: 22 - protein, 105 aa, Artificial sequence
  Synthetic sequence
ELESGGGLVPGSLLCASGFSFSLSFYMCWVRQAPGKGLEWIACIYSGSSGSTYYASWAKGRFTISKSTVLQMLADTAYFCARSASSTTFHYFNLWGQGTLVTVSS

SEQ ID NO: 23 - protein, 5 aa, Artificial sequence
  Synthetic sequence
GGXGG

SEQ ID NO: 24 - protein, 6 aa, Artificial sequence
  Synthetic sequence
DTSKNQ

SEQ ID NO: 25 - protein, 6 aa, Artificial sequence
  Synthetic sequence
DNSKNT

SEQ ID NO: 26 - protein, 6 aa, Artificial sequence
  Synthetic sequence
DNAKNS

SEQ ID NO: 27 - protein, 6 aa, Artificial sequence
  Synthetic sequence
DDSKNS

SEQ ID NO: 28 - protein, 6 aa, Artificial sequence
  Synthetic sequence
DDSKNT

SEQ ID NO: 29 - protein, 6 aa, Artificial sequence
  Synthetic sequence
DESTST

SEQ ID NO: 30 - protein, 6 aa, Artificial sequence
  Synthetic sequence
DGSKSI

SEQ ID NO: 31 - protein, 6 aa, Artificial sequence
  Synthetic sequence
DKSIST

SEQ ID NO: 32 - protein, 6 aa, Artificial sequence
  Synthetic sequence
DKSKNQ

SEQ ID NO: 33 - protein, 6 aa, Artificial sequence
  Synthetic sequence
DKSTST

SEQ ID NO: 34 - protein, 6 aa, Artificial sequence
  Synthetic sequence
DMSTST

SEQ ID NO: 35 - protein, 6 aa, Artificial sequence
  Synthetic sequence
DNAKNT

SEQ ID NO: 36 - protein, 6 aa, Artificial sequence
  Synthetic sequence
DNSKNS

SEQ ID NO: 37 - protein, 6 aa, Artificial sequence
  Synthetic sequence
DRSKNQ

SEQ ID NO: 38 - protein, 6 aa, Artificial sequence
  Synthetic sequence
DRSMST

SEQ ID NO: 39 - protein, 6 aa, Artificial sequence
  Synthetic sequence
DTSAST

SEQ ID NO: 40 - protein, 6 aa, Artificial sequence
  Synthetic sequence
DTSIST

SEQ ID NO: 41 - protein, 6 aa, Artificial sequence
  Synthetic sequence
DTSKSQ

SEQ ID NO: 42 - protein, 6 aa, Artificial sequence
  Synthetic sequence
DTSTDT

SEQ ID NO: 43 - protein, 6 aa, Artificial sequence
  Synthetic sequence
DTSTST

SEQ ID NO: 44 - protein, 6 aa, Artificial sequence
  Synthetic sequence
DTSVST

SEQ ID NO: 45 - protein, 6 aa, Artificial sequence
  Synthetic sequence
ENAKNS

SEQ ID NO: 46 - protein, 6 aa, Artificial sequence
  Synthetic sequence
NTSIST

SEQ ID NO: 47 - protein, 7 aa, Artificial sequence
  Synthetic sequence
SLQPEDF

SEQ ID NO: 48 - protein, 7 aa, Artificial sequence
  Synthetic sequence
RVEAEDV

SEQ ID NO: 49 - protein, 7 aa, Artificial sequence
  Synthetic sequence
NIESEDA

SEQ ID NO: 50 - protein, 7 aa, Artificial sequence
  Synthetic sequence
RLEPEDF

SEQ ID NO: 51 - protein, 7 aa, Artificial sequence
  Synthetic sequence
SLEAEDA

SEQ ID NO: 52 - protein, 7 aa, Artificial sequence
  Synthetic sequence
SLEPEDF

SEQ ID NO: 53 - protein, 7 aa, Artificial sequence
  Synthetic sequence
SLQAEDV

SEQ ID NO: 54 - protein, 7 aa, Artificial sequence
  Synthetic sequence
SLQPDDF

SEQ ID NO: 55 - protein, 7 aa, Artificial sequence
  Synthetic sequence
SLQPEDI

SEQ ID NO: 56 - protein, 7 aa, Artificial sequence
  Synthetic sequence
SLQPEDV

SEQ ID NO: 57 - protein, 7 aa, Artificial sequence
  Synthetic sequence
SLQSEDF

SEQ ID NO: 58 - protein, 19 aa, Artificial sequence
  Synthetic sequence
MEFGLSWVFLVAILKGVQC

SEQ ID NO: 59 - DNA, 24 bp, Artificial sequence
  Synthetic sequence
tcgcactcaacacagacgctcacc

SEQ ID NO: 60 - DNA, 24 bp, Artificial sequence
  Synthetic sequence
atggagactgggctgcgctggctt

SEQ ID NO: 61 - DNA, 25 bp, Artificial sequence
  Synthetic sequence
gctcagcgagtagaggcctgaggac

SEQ ID NO: 62 - DNA, 25 bp, Artificial sequence
  Synthetic sequence
ttggggggaagatgaagacagacgg

SEQ ID NO: 63 - DNA, 24 bp, Artificial sequence
  Synthetic sequence
cagtgcaggcaggacccagcatgg

SEQ ID NO: 64 - DNA, 23 bp, Artificial sequence
  Synthetic sequence
gccctggcaggcgtctcrctcta

SEQ ID NO: 65 - protein, 7 aa, Artificial sequence
  Synthetic sequence
DLECADA

SEQ ID NO: 66 - protein, 7 aa, Artificial sequence
  Synthetic sequence
SLQPDDA

SEQ ID NO: 67 - protein, 7 aa, Artificial sequence
  Synthetic sequence
SLQPDDF